SEQUENCE LISTING (3 sequences)

SEQ ID NO: 1 - protein, 298 aa, Homo sapiens
MSLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQGIREVILCKDQDGKIGLRLKSIDNGIFVQLVQANSPASLVGLRFGDQVLQINGENCAGWSSDKAHKVLKQAFGEKITMTIRDRPFERTITMHKDSTGHVGFIFKNGKITSIVKDSSAARNGLLTEHNICEINGQNVIGLKDSQIADILSTSGTVVTITIMPAFIFEHIIKRMAPSIMKSLMDHTIPEV

SEQ ID NO: 2 - protein, 245 aa, Homo sapiens
MEKTELIQKAKLAEQAERYDDMATCMKAVTEQGAELSNEERNLLSVAYKNVVGGRRSAWRVISSIEQKTDTSDKKLQLIKDYREKVESELRSICTTVLELLDKYLIANATNPESKVFYLKMKGDYFRYLAEVACGDDRKQTIDNSQGAYQEAFDISKKEMQPTHPIRLGLALNFSVFYYEILNNPELACTLAKTAFDEAIAELDTLNEDSYKDSTLIMQLLRDNLTLWTSDSAGEECDAAEGAEN

SEQ ID NO: 3 - protein, 417 aa, Homo sapiens
MSLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNI

(19) United States
(12) Patent Application Publication
(10) Pub. No.: US 2016/0011213 A1
(43) Pub. Date: Jan. 14, 2016

(54) ASSAY METHOD

(71) Applicant: ISIS INNOVATION LIMITED, Oxford, Oxfordshire (GB)

(72) Inventor: George Tofaris, Oxford (GB)

(73) Assignee: ISIS INNOVATION LIMITED, Oxford (GB)

(21) Appl. No.: 14/773,206

(22) PCT Filed: Mar. 5, 2014
(86) PCT No.: PCT/GB2014/050647

§ 371 Date: Sep. 4, 2015

(30) Foreign Application Priority Data
Mar. 5, 2013 (GB) 1303906.0

(51) Int. Cl. G01N 33/68; A61K 38/45; A61K 38/44; A61K 38/17

(52) U.S. Cl. CPC G01N 33/6896; A61K 38/17; A61K 38/45; A61K 38/44; G01N 2800/2835; G01N 2800/52; G01N 2333/4703; G01N 2333/91225

(57) ABSTRACT

A method for the identification or monitoring of Parkinson's Disease (PD) in an individual, which method comprises measuring the level of at least one marker in a sample taken from the individual.

The present invention relates to a method for the identification or monitoring of Parkinson's disease in an individual.

### BACKGROUND

Parkinson's disease (PD), which causes significant disability and loss of quality of life, is the second most common neurodegenerative disorder in the world. Identification of Parkinson's disease in an individual, especially in the early stage, is of great importance. However, early identification can be challenging, as the signs and symptoms overlap with other syndromes.

There is a particular need for methods which allow the identification of patients at the pre-motor stage of PD before the majority of dopaminergic neurons have degenerated. There is also a need for methods which allow the identification of a patient as having PD as opposed to another neurodegenerative disorder. There is also a need for methods which allow the monitoring of PD progression in response to neuroprotective therapies.

### SUMMARY OF THE INVENTION

The present inventors have identified proteins which are differentially expressed in PD patients relative to individuals without PD and particularly relative to individuals with a neurodegenerative disorder which is not PD. These proteins may therefore be used as markers for the diagnosis or monitoring of PD. The proteins which may be used as markers are shown in Table 1 below, and are listed as follows: Syntenin-1, 14-3-3 theta, phosphoglycerate kinase 1 (PGK1), Programmed cell death protein 6, Complement factor B, Guanine nucleotide-binding protein G(i) subunit alpha-2, Fermitin family homolog 3, Alpha-2-macroglobulin, Integrin beta-1, Complement factor H, beta-actin, Glyceraldehyde-3-phosphate dehydrogenase, Casein kinase II subunit alpha, Prostaglandin E synthase 3, Glucose transporter type 1 (GLUT-1), POTE ankyrin domain family member J; 14-3-3 gamma, and Guanine nucleotide-binding protein G(k) subunit alpha.

The differential expression of these proteins is particularly apparent in exosomes isolated from the blood serum of individuals. Exosomes are membrane vesicles that may be secreted by all mammalian cell types, including neurons. They are naturally occurring at low levels in body fluids, suggesting a role in cell-cell or organ-organ communication. Exosomes are typically less than 200 nm in diameter. They are generated by an inward budding of the limiting membrane of multivesicular bodies, leading to entrapment of a small portion of the cytosol in intraluminal vesicles. By examining the contents of the cytosol of exosomes the inventors have found that it is possible to identify and monitor markers of the cellular changes that occur in pathological states, such as PD.

The present invention provides a method for the identification or monitoring of Parkinson's Disease (PD) in an individual, which method comprises measuring the level of at least one marker in a first sample taken from the individual, wherein said at least one marker is selected from the proteins of Table 1, and wherein if the level of 14-3-3 theta is measured said sample is not taken from the cerebral cortex of the individual. In said method, the at least one marker is preferably Syntenin-1, 14-3-3 theta or phosphoglycerate kinase 1 (PGK1), and is particularly preferably Syntenin-1.

The invention also provides a method for the treatment of PD in an individual, which method comprises identifying the individual as having PD in accordance with a method of the invention and subsequently administering a therapeutically effective amount of a treatment agent to the individual.

The invention also provides a method for determining the effect of a treatment agent on the progression of PD, which method comprises monitoring the progression of PD in an individual in accordance with a method of the invention, wherein the first sample is taken from the individual prior to the administration of a treatment agent and the second sample is taken from the individual after the administration of a treatment agent.

The invention also provides a method for the treatment of PD in an individual, which method comprises administering to the individual a composition comprising microvesicles, wherein said microvesicles contain at least one protein of Table 1, or which method comprises stimulating the endogenous production of said microvesicles.

The invention also provides a composition comprising microvesicles for use in a method of treating PD, wherein said microvesicles contain at least one protein of Table 1.

The invention also provides the use of microvesicles containing at least one protein of Table 1 in the manufacture of a medicament for the treatment of PD.

### BRIEF DESCRIPTION OF THE FIGURES

FIG. 1A Nanotraking analysis (NTA) of serum microvesicles which reveals a major peak at the size corresponding to exosomes isolated from NSC34 conditioned media whereas spike-in experiments shows a further increment in microvesicles of the same size.

FIG. 1B. Immunoblots which confirm the presence of the exosome markers flotillin1 and Tsg101 both in cell conditioned media and serum preparations

FIG. 1C. Electron microscopy images which show membrane-bound vesicles in serum preparations.

FIGS. 1D and E show the mean number (panel D) and size (panel E) of isolated microvesicles in individually extracted patient samples. No difference was observed in the mean number or size (n=20 per group).

FIG. 2 is a Venn diagram showing protein overlap between albumin/immunoglobulin depleted serum (“depleted serum”), serum microvesicles and human cell lysates. Serum microvesicles display a distinct proteomic profile.

FIGS. 3A and B show the results of Principal Component Analysis (PCA) of analysed samples. Label-free relative protein quantitation of all samples was performed and ANOVA p values were calculated after grouping of the samples into healthy controls and PD (incl. one PD group with a heterozygous GBA mutation) to identify the proteins that are detected with significantly differential abundances (143 proteins, ANOVA p<0.05). Panel A shows the separation of the analyzed samples (blue dots) and the loadings of the 143 proteins (red dots). Syntenin-1 is indicated by *; 14-3-3 theta is indicated by **). Panel B shows that 38 proteins out of the 143 protein identified in panel A can further separate PD and MND samples (ANOVA p<0.05, except 14-3-3 theta, p=0.08). Panel C shows immunoblots with specific antibodies against syntenin 1 and 14-3-3 theta, which confirm the results of the PCA in a separate group of early PD patients (Hoehn & Yahr (H&Y) stage 1 or 2) compared to controls (n=60 PD patients and 30 controls, *p<0.02, t-test). Flotillin was also detected as a positive control. Panel D shows the collated results for the abundance of each protein in Controls versus PD patient groups. Protein abundance corrected for exosome number.

FIG. 4. Primary rat neurons were placed in neurobasal medium lacking B27 at day eight in culture. Microvesicles or liposomes were quantified by NTA. Equal vesicle numbers from PD patients or controls were added 5 h after nutrient deprivation (ND) and incubated for further 18 h (n=30 individually prepared patient or control-derived microvesicle preparations). Panel A shows that neuronal metabolic activity as assessed by the MTT assay, was increased upon nutrient deprivation and normalised in neurons treated with PD-derived microvesicles but not liposomes or controls. Panel B shows that Caspase 3 activation was significantly reduced in neurons treated with PD-derived microvesicles compared to controls. Panel C shows representative images of caspase-3 and β-III tubulin doubly positive neurons under different experimental conditions.

### DETAILED DESCRIPTION

The present invention concerns methods for the identification and monitoring of Parkinson's Disease (PD) in an individual. The methods of the invention involve measuring the level of at least one marker in a sample taken from the individual. The at least one marker is selected from the proteins shown in Table 1.

TABLE 1

| UNIPROT | | |
|---|---|---|
| Accession | Version | Description |
| O00560 | 134 | Syntenin-1 |
| P27348 | 141 | 14-3-3 protein theta |
| P00558 | 164 | Phosphoglycerate kinase 1 |
| O75340 | 131 | Programmed cell death protein 6 |
| P00751 | 189 | Complement factor B |
| P04899 | 162 | Guanine nucleotide-binding protein |
| | | G(i) subunit alpha-2 |
| Q86UX7 | 106 | Fermitin family homolog 3 |
| P01023 | 167 | Alpha-2-macroglobulin |
| P05556 | 179 | Integrin beta-1 |
| P08603 | 177 | Complement factor H |
| P60709 | 126 | Actin, cytoplasmic 1 (Beta-actin) |
| P04406 | 189 | Glyceraldehyde-3-phosphate dehydrogenase |
| | | (GAPDH) |
| P68400 | 116 | Casein kinase II subunit alpha |
| Q15185 | 136 | Prostaglandin E synthase 3 |
| P11166 | 172 | Solute carrier family 2, facilitated glucose |
| | | transporter memb 1, (GLUT-1) |
| P0CG39 | 32 | POTE ankyrin domain family member J |
| P61981 | 117 | 14-3-3 protein gamma |
| P08754 | 164 | Guanine nucleotide-binding protein G(k) |
| | | subunit alpha |

The at least one marker is preferably selected from Syntenin-1, 14-3-3 theta and phosphoglycerate kinase 1 (PGK1), and is particularly preferably Syntenin-1. Where 14-3-3 theta is measured, the sample is not taken from the cerebral cortex of the individual. The sequences of Syntenin-1, 14-3-3theta and PGK1 are shown in full below.

Human Syntenin-1 has the sequence:
MSLYPSLEDL KVDKVIQAQT AFSANPANPA ILSEASAPIP
HDGNLYPRLY PELSQYMGLS LNEEEIRANV AVVSGAPLQG
QLVARPSSIN YMVAPVTGND VGIRRAEIKQ GIREVILCKD
QDGKIGLRLK SIDNGIFVQL VQANSPASLV GLRFGDQVLQ
INGENCAGWS SDKAHKVLKQ AFGEKITMTI RDRPFERTIT
MHKDSTGHVG FIFKNGKITS IVKDSSAARN GLLTEHNICE
INGQNVIGLK DSQIADILST SGTVVTITIM PAFIFEHIIK
RMAPSIMKSL MDHTIPEV
[UNIPROT Accession no: O00560 v134]
Human 14-3-3 theta has the sequence:
MEKTELIQKA KLAEQAERYD DMATCMKAVT EQGAELSNEE
RNLLSVAYKN VVGGRRSAWR VISSIEQKTD TSDKKLQLIK
DYREKVESEL RSICTTVLEL LDKYLIANAT NPESKVFYLK
MKGDYFRYLA EVACGDDRKQ TIDNSQGAYQ EAFDISKKEM
QPTHPIRLGL ALNFSVFYYE ILNNPELACT LAKTAFDEAI
AELDTLNEDS YKDSTLIMQL LRDNLTLWTS DSAGEECDAA
EGAEN
[UNIPROT Accession no: P27348 v141]
Human phosphoglycerate kinase 1 (PGK1)
MSLSNKLTLD KLDVKGKRVV MRVDFNVPMK NNQITNNQRI
KAAVPSIKFC LDNGAKSVVL MSHLGRPDGV PMPDKYSLEP
VAVELKSLLG KDVLFLKDCV GPEVEKACAN PAAGSVILLE
NLRFHVEEEG KGKDASGNKV KAEPAKIEAF RASLSKLGDV
YVNDAFGTAH RAHSSMVGVN LPQKAGGFLM KKELNYFAKA
LESPERPFLA ILGGAKVADK IQLINNMLDK VNEMIIGGGM
AFTFLKVLNN MEIGTSLFDE EGAKIVKDLM SKAEKNGVKI
TLPVDFVTAD KFDENAKTGQ ATVASGIPAG WMGLDCGPES
SKKYAEAVTR AKQIVWNGPV GVFEWEAFAR GTKALMDEVV
KATSRGCITI IGGGDTATCC AKWNTEDKVS HVSTGGGASL
ELLEGKVLPG VDALSNI
[UNIPROT Accession no: P00558 v164]

The individual may be a human or an animal, and is typically a human. The individual is typically suspected of being at risk of developing a neurodegenerative disorder, particularly PD. This may be because the individual has a familial history of such a disorder, or because the individual presents with one or more symptoms associated with a clinical diagnosis of such a disorder.

The individual typically does not exhibit any of the primary motor symptoms associated with PD. Preferably the individual does not exhibit any of the primary or secondary motor symptoms associated with PD. The individual may exhibit one or more of the non-motor symptoms associated with PD, which are known to precede the motor features of PD by several years (typically by 7-10 years). Such symptoms include hyposmia or REM sleep behavioural disorder. However, the individual may exhibit no symptoms associated with a clinical diagnosis of a neurodegenerative disorder, particularly PD.

Accordingly, the individual may exhibit none, or one or more of the following symptoms associated with PD:

### Primary Motor Symptoms

(i) Tremor: About 70 percent of people with Parkinson's experience tremor, which is usually apparent when muscles are relaxed (it is a “resting tremor”). This is often the first identifiable symptom. The tremor is typically in either the hand or foot on one side of the body, or less commonly in the jaw or face. The tremor often spreads to the other side of the body as the disease progresses, but remains most apparent on the original side of occurrence.

(ii) Bradykinesia (Slow movement): the patient displays markedly slow movement. In addition to slow movement, a person with bradykinesia will typically also have incomplete movement, difficulty initiating movements and difficulty in suddenly stopping ongoing movements. People who have bradykinesia may walk with short, shuffling steps (festination). Bradykinesia and rigidity can occur in the facial muscles, reducing a person's range of facial expressions and resulting in a “mask-like” appearance.

(iii) Rigidity: also called increased muscle tone, means stiffness or inflexibility of the muscles. In rigidity, the muscle tone of an affected limb is always stiff and does not relax, sometimes resulting in a decreased range of motion. For example, a person who has rigidity may not be able to swing his or her arms when walking because the muscles are too tight. Rigidity can cause pain and cramping.

(iv) Postural Instability (Impaired Balance and Coordination): Subjects with PD often experience instability when standing, or have impaired balance and coordination. The subject may go through periods of “freezing,” in which the subject finds it difficult to start walking. Slowness and incompleteness of movement can also affect speaking and swallowing.

### Secondary Motor Symptoms

Not all PD subjects will experience secondary motor symptoms. However, most subjects typically exhibit one or more of the following: Stooped posture, a tendency to lean forward (camptocormia); Dystonia; Impaired fine motor dexterity and motor coordination; Impaired gross motor coordination; Akathisia; Speech problems, such as softness of voice or slurred speech caused by lack of muscle control; Loss of facial expression, or “masking”; Micrographia (small, cramped handwriting); Difficulty swallowing.

### Non-Motor Symptoms

A number of non-motor symptoms are associated with PD. However, these symptoms are not specific for PD, and are typically only identified as indicating PD retrospectively. That is, in the absence of another indicator (such that provided by the method of the invention) the non-motor symptoms experienced by a subject are not typically recognised as indicating PD until after the presence of motor symptoms has been confirmed by a specialist. Even so, a PD patient will typically exhibit one or more of the following: Pain; Dementia; Sleep disturbances (e.g. REM sleep behaviour disorder (RBD)); Hyposmia; autonomic features such as constipation, urinary urgency and sexual dysfunction; Skin problems; Depression or anxiety; slowed thinking (bradyphrenia); Fatigue and aching.

The individual may or may not have been categorised according to the Hoehn-Yahr scale or the modified Hoehn-Yahr scale. The Hoehn-Yahr scale is a commonly used system for describing how the symptoms of Parkinson's disease progress. The scale allocates stages from 0 to 5 to indicate the relative level of disability. The modified Hoehn-Yahr scale includes the additional stages 1.5 and 2.5 to help describe the intermediate course of the disease. If categorised, the individual is typically grade 2 or lower. The stages of both scales are shown below in Table 2.

TABLE 2

| Stage | Hoehn-Yahr scale | Modified Hoehn-Yahr scale |
|---|---|---|
| 1 | Unilateral involvement only | Unilateral involvement only |
| | usually with minimal or no | |
| | functional disability | |
| 1.5 | — | Unilateral and axial involvement |
| 2 | Bilateral or midline involvement | Bilateral involvement without |
| | without impairment of balance | impairment of balance |
| 2.5 | — | Mild bilateral disease with |
| | | recovery on pull test |
| 3 | Mild to moderate bilateral | Mild to moderate bilateral |
| | disease; some postural insta- | disease; some postural insta- |
| | bility; physically independent | bility; physically independent |
| 4 | Severe disability; still able | Severe disability; still able |
| | to walk or stand unassisted | to walk or stand unassisted |
| 5 | Wheelchair bound or bedridden | Wheelchair bound or bedridden |
| | unless aided | unless aided |

Determining the level of a marker in a sample may be achieved by any suitable method. A preferred method is an immunoassay such as an ELISA or any suitable electrochemical detection method. Some suitable methods are shown in the Examples. The method may typically involve an agent which is capable of binding specifically to a given marker, such as an agent capable of specifically binding to any one of the proteins shown in Table 1. The agent may preferably be an antibody, or antigen binding fragment thereof, which binds specifically to said marker. For example the agent may be an antibody, or antigen binding fragment thereof, which binds specifically to Syntenin-1, 14-3-3 theta or PGK1. By specific binding, it will be understood that the agent binds to its target with no significant cross-reactivity to any other molecule, particularly any other protein. Cross-reactivity may be assessed by any suitable method.

The sample is typically a biological fluid. A biological fluid may be a fluid that has been obtained from an individual. The biological fluid may be selected from blood, blood serum, urine, tears, saliva, sweat, and cerebrospinal fluid. The biological fluid sample is typically a blood serum sample. The biological fluid may be undiluted, meaning that it has not been diluted with another liquid. Optionally, the sample may comprise a biological fluid obtained from a subject, e.g. a human or animal, and a diluent. The sample may be fresh or may be preserved, e.g. frozen, prior to use.

In a preferred embodiment, the level of at least one of marker is determined by measuring the level of said marker in exosomes isolated from said sample. Exosomes are membrane vesicles that may be secreted by all mammalian cell types, including neurons. They may be naturally occurring at low levels in body fluids, suggesting a role in cell-cell or organ-organ communication. Exosomes are typically less than 200 nm in diameter. For example, exosomes typically range in diameter from 40 to 150 nm in diameter. They are generated by an inward budding of the limiting membrane of multivesicular bodies, leading to entrapment of a small portion of the cytosol in intraluminal vesicles.

Any suitable method may be used to isolate exosomes from biological fluids. Such methods may be carried out such that the samples remain at around 4° C. throughout. Exosomes may be isolated from a sample by centrifugation and/or filtration. Alternative methods may involve microfluidic isolation of exosomes from a sample. Centrifugation methods typically include multiple rounds of differential centrifugation, followed by ultracentrifugation with or without filtration.

A preferred method involves a differential centrifugation phase involving at least three rounds of serial centrifugation at increasing centrifugal force, in which the supernatant is recovered after each round and subjected to the next round. After the differential centrifugation phase, the final supernatant is subjected to an ultracentrifugation phase. This phase involves at least two rounds of ultracentrifugation, each of which may also include filtration. Following a first round of ultracentrifugation with filtration, the pellet is typically collected, resuspended and subjected to the subsequent round of ultracentrifugation, before collection and resuspension of the pellet, which typically contains the exosomes and can be resuspended for further analysis.

A method of this type can be illustrated by the following example. A sample of biological fluid is subjected to a first round of centrifugation at approximately 800 g for around 10 minutes. The supernatant from this step is subjected to a second round of centrifugation at approximately 1500 g for around 10 minutes. The supernatant from this step is subjected to a third round of centrifugation at approximately 17000 g for around 15 minutes. The supernatant from this step is then filtered, typically through a 0.2 μm filter spun in an ultracentrifuge at approximately 160000 g for around 1 hour. The supernatant from this step is removed and the pellet resuspended before being spun in an ultracentrifuge at approximately 160000 g for around 1 hour. The supernatant from this step is removed and the pellet containing exosomes is resuspended for further analysis. A method of this type is also set out in the Examples.

Protocols of this type effectively overcome a major challenge in the analysis of complex proteomes, that is contamination with highly abundant serum proteins.

The methods of the invention may involve comparing the level of a given marker, such as a protein of Table 1 (for example Syntenin-1, 14-3-3 theta or PGK1), in a particular sample to another level, such as a control level of the said marker or the level of the said marker in a second sample taken from the same individual. In this context, it will be understood that the level of a given marker is compared to a control level of the same marker or the level of the same marker in a second sample taken from the same individual.

In the methods of the invention, the level of a given marker in a sample may be compared directly to another level of the said marker, or may first be normalised against the level of a different protein such as a ubiquitous exosomal protein, for example flotillin. That is, the method may involve calculating the ratio of the level of a given marker in sample to the level of a ubiquitous exosomal protein, for example flotillin, in the same sample. For example, the ratio of Syntenin-1:flotillin, 14-3-3 theta:flotillin, or PGK-1: flotillin may be determined for a sample. Such a normalised level or ratio may then be compared to a control value for said normalised level or ratio, or to the corresponding normalised level or ratio for a second sample taken from the same individual.

Irrespective of whether the level of a marker is compared directly or following normalisation or calculation of a ratio, in the course of such a comparison, it may be determined whether the level of a marker in a sample is higher or lower than the level to which it is compared. A level of a marker, such as a protein of Table 1, may be determined to be higher than a level to which it is compared if it is at least 1.1-fold, 1.2-fold, 1.5-fold, 1.75 fold, 2-fold, 3-fold, 4-fold, 5-fold or 10-fold higher than the level to which it is compared. A level of a marker, such as a protein of Table 1, may be determined to be lower than a level to which it is compared if it is at least 1.1-fold, 1.2-fold, 1.5-fold, 1.75 fold, 2-fold, 3-fold, 4-fold, 5-fold or 10-fold lower than the level to which it is compared.

A method of the invention may be used to identify whether or not an individual has PD. The method may involve comparing the level of at least one marker selected from the proteins of Table 1 (for example Syntenin-1, 14-3-3 theta or PGK1) in a sample taken from the individual to a control level. An individual may be identified as having PD if the level of the marker in said sample is higher than said control level. An individual may be identified as not having PD if the level of the marker in said sample is lower than said control level.

A control level for a marker may be the level of the marker in a sample taken from an individual not suffering from PD. The individual not suffering from PD is typically healthy, and is preferably matched for age, sex and co-morbidity with the individual that is the subject of the method. The individual not suffering from PD preferably displays none of the motor symptoms associated with PD, and most preferably displays no symptoms associated with PD.

The method of the invention may also be used to distinguish between PD in an individual and another neurodegenerative disorder, which is not PD. The individual may be suspected of having a neurodegenerative disorder, which may be PD but may be another neurodegenerative disorder. Neurodegenerative disorders which are not PD include motorneurone disease (MND), Alzheimers Disease (AD), Multisystem atrophy or Fronto-temporal Dementia (FTD).

The individual may be suspected of having PD or another neurodegenerative disorder based on the physical symptoms with which they present. For example, the individual may present one or more symptoms which overlap between PD and other neurodegenerative disorders. In this embodiment, the method of the invention typically comprises comparing the level of a marker in a sample taken from the individual to a control level. The control level in this embodiment may be the level of said marker measured in a sample taken from an individual having said neurodegenerative disorder which is not PD, or from a healthy individual as defined above.

The individual may be identified as having PD and not another neurodegenerative disorder which is not PD if the level of at least one marker in said sample is higher than the control level. The individual may be identified as having a neurodegenerative disorder which is not PD if the level of said at least one marker in said sample is lower than the control level.

The method of the invention may include subsequently treating an individual identified as having PD by a method as described herein. Treating an individual typically comprises administering a therapeutically effective amount of a treatment agent to the individual. The treatment agent is typically a neuroprotective agent, which may be selected from L-Dopa, an anti-apoptotic, an anti-oxidant, an anti-glutamatergic, a monoamine oxidase B inhibitor, an adenosine antagonist, a dopamine agonist, a mitochondrial stabiliser, a promoter of alpha-synuclein clearance or a trophic factor. For example, the agent may be rasagiline, selegiline, ropinirole, pramipexole, nicotine, minocycline, creatine, caffeine, or coenzyme Q10.

A method of the invention may also be used to monitor the progression of PD in an individual. That is, the method may be used to determine whether the PD of an individual is worsening or improving over time. The individual may have previously been diagnosed with PD.

Where the progression of PD is to be monitored, typically, samples are taken from the same patient over a period of time to monitor the progression of their Parkinson's disease. For example, the method may be conducted on samples taken from the individual at intervals of 1 month, 2 months, 3 months, 6 months, 1 year, 18 months, 2 years or 3 years or more. Alternatively, the levels are compared to a control level as defined above, or to samples from other individuals known to be suffering from PD to correlate the level of a given marker with a particular stage of PD.

The method may typically involve comparing the level of at least one marker selected from the proteins of Table 1 in a first sample taken from an individual to the level of said marker in a second sample taken from the same individual at a later time. The PD of the individual may be identified as worsening or improving if there is a higher level of the marker in the second sample relative to the first sample. The PD of the individual may be identified as improving or worsening if there is a lower level of the marker in the second sample relative to the first sample.

A higher level or an increase in the level of said at least one marker may typically be associated with the worsening of PD in an individual, for example the progression from stage 1 to stage 2 and stage 2.5 PD based on the Hoehn-Yahr scale or modified Hoehn-Yahr scale or the progression to dementia and/or disability. Alternatively it may lead to classifying a patient as being at higher risk of such a progression. However, a higher level or an increase in the level of a said marker, may be associated with the improvement of PD or reduced risk of progression, particularly where a said marker may have a protective effect.

A lower level or a decrease in the level of said at least one marker may typically be associated with improvement of PD of an individual. Alternatively it may lead to classifying the individual as being at lower risk of progression to a more severe stage of PD. However, a lower level or a decrease in the level of a said marker, may be associated with the worsening of PD or an increased risk of progression, particularly where a said marker may have a protective effect.

The method of the invention may also be used to determine the effect of a treatment agent on the progression of PD in an individual, for example one of the treatment agents described above. In this embodiment, the progression of PD is monitored in accordance with a method as described above, with the level of at least one marker selected from the proteins of Table 1 in a sample taken before administration of the treatment agent being compared to the level of said marker in a sample taken after administration of the treatment agent. A decrease or increase in the level of said marker in the second sample relative to the first sample indicates that the treatment agent has had a positive effect on disease progression. An increase or decrease in the level of said marker in the second sample relative to the first sample indicates that the treatment agent has had no or a negative effect on disease progression.

This embodiment may further comprise altering the treatment regime of the individual based on the result of the method. This may comprise altering the dose of the existing treatment agent, and/or administering an alternative treatment agent to the individual. For example, in the event of no or a negative effect on disease progression (that is, PD is unchanged or worsening), the method may comprise increasing the dose of the treatment agent, and/or administering an alternative treatment agent to the individual. The alternative treatment agent may carry a higher risk of adverse side-effects. In the event of a positive effect on disease progression (that is, PD is improving), the method may comprise decreasing the dose of the treatment agent, and/or administering an alternative treatment agent to the individual. The alternative treatment agent in this instance typically carries a lower risk of adverse side-effects. It is well-known in the art that certain treatment agents for PD carry a risk of adverse side-effects. For example, L-dopa can give rise to dyskinesias and dopamine agonists can give rise to impulse control disorders.

The invention also provides a method for the treatment of PD in an individual, which method comprises administering a composition comprising microvesicles which contain at least one protein of Table 1 (preferably selected from Syntenin-1, 14-3-3 theta and PGK1) to the individual, or stimulating endogenous production of said microvesicles in the individual. A said microvesicle is typically enriched in said at least one protein, meaning that the protein is present in relatively high abundance when compared to exosomes from healthy controls. A said microvesicle may be any suitable microvesicle, such as a synthetic liposome. A said microvesicle may be formulated as a composition with one or more pharmaceutically acceptable diluents or carriers. The microvesicle or composition thereof may be formulated for administration directly to the nervous system or brain of an individual, for example by intrathecal injection into the cerebrospinal fluid or by intravenous administration into the systemic circulation.

The invention also provides a said composition for use in a method of treating PD, or a said microvesicle for use in the manufacture of a medicament for the treatment of PD.

The invention also provides a synthetic liposome containing at least one protein of Table 1. Methods for the production of synthetic liposomes are known in the art. The following Examples illustrate the invention:

### Example 1

### Sample Populations

Samples were obtained from patients and controls that were enrolled in the Oxford PD Cohort, a prospective study of patients within the first three years of their diagnosis.

Serum samples were collected from the following groups of age-matched individuals:
NC: Controls: 3 groups of 12 per group, mean age 64.36;

17 Male, 19 Female;

IPD: Idiopathic PD subjects: 3 groups of 12 per group, mean age 64.61;

18 Male, 18 Female

PD^{GBA}: PD subjects with GBA mutation: 1 group of 13 subjects, mean age 62.15;

7 Male, 6 Female

The samples were stored at −80° C. in accordance with standard protocols. In brief: 10 ml of blood was collected from all subjects that participated in the study and processed during the first consultation. The blood was allowed to clot at room temperature for 10 minutes then centrifuged for 10 minutes at 1300 g. The serum supernatant was aliquoted into cryovial with 0.75 ml of serum placed into each tube and placed immediately on dry ice until stored in −80° C. Stored serum samples from PD patients and aged matched controls were thawed from −80° C. on ice and pooled into separate groups as outlined below. Where MND patients are discussed in the following experiments, the MND patient samples were obtained from the BioMOx Cohort and were processed as above. However, because smaller serum volumes were available for this group, 0.4 ml of serum was used from 22 individual patients (Average age 65 years; 14 male; 8 female).

### Microvesicle Isolation

Stored serum samples were thawed from −80° C. 1.3 ml of serum from each subject in the groups described above was pooled to create a pooled sample for each group of 12 subjects. For each of the three pooled samples, the following protocol was followed:
First centrifugation round: 800 g for 10 minutes at 4° C.
Recover supernatatnt and subject to:
Second centrifugation round: 1500 g for 10 minutes at 4° C.;
Recover supernatatnt and subject to:
Third centrifugation round: 17000 g for 15 minutes at 4° C.
Recover supernatant and subject to:
Filtration through a 0.2 μm filter spun in an ultracentrifuge at 160000 g for 1 hour at 4° C. (first ultracentrifugation round);
Remove supernatant, recover and resuspend pellet in 5 ml of Hanks Balanced Salt Solution (HBSS), subject to:
Second ultracentrifugation round: at 160000 g for 1 hour at 4° C.;
Remove supernatant, recover and resuspend pellet in HBSS for subsequent analysis.
   The above protocol effectively overcomes a major challenge in the analysis of complex proteomes, that is contamination with highly abundant serum proteins. This was verified by separating representative samples by gel electrophereis with subsequent Coomassie staining. Highly abundant serum proteins were demonstrably removed (data not shown).

### Characterisation of Microvesicles

The purified microvesicles were characterised with nanotracking analysis, electron microscopy and immunoblotting. These data showed that the majority of isolated microvesicles have properties that are characteristic of exosomes.

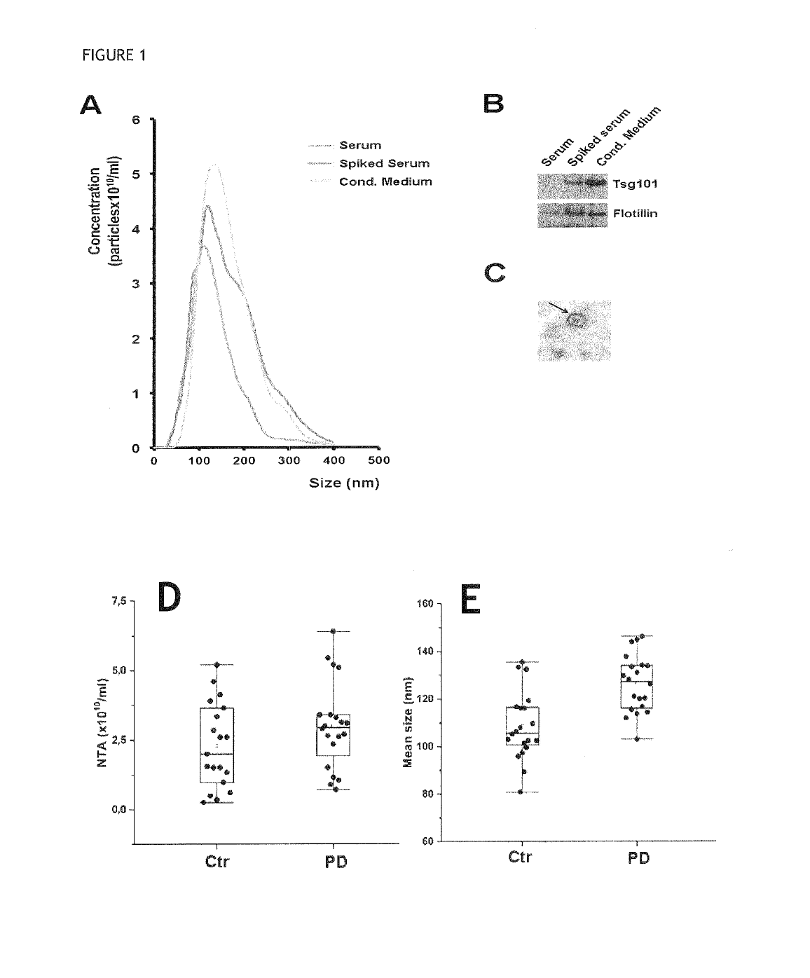

The results of the nanotracking analysis (NTA) are shown in FIG. 1A. There is a major peak at the size corresponding to exosomes isolated from NSC34 cell-conditioned media (as a positive control). In addition, immunoblotting (see FIG. 1B) confirmed the presence of the exosome markers flotillin and TSG101 in both cell-conditioned media and serum preparations. To confirm that these two microvesicle populations are similar we spiked serum with exosomes derived from NSC34 and showed that this led to a further increase in the corresponding size (see FIG. 1A) and protein markers (see FIG. 1B). Finally, electron microscopy was used to show the membrane-bound vesicles in serum preparations (see FIG. 1C). Initial analysis suggested that there is no difference in microvesicle number or size between PD patients and healthy controls (n=20 per sample, FIG. 1D, E).

The protocol for the nanotracking analysis was as follows: Microvesicle size and concentration were assessed using a NS500 instrument (Nanosight Ltd. Amesbury, UK) equipped with a 405 nm laser and a CMOS camera, as previously described (Gardiner et al., 2013). Briefly, samples were diluted in filtered PBS immediately prior to use. 5×30 second videos were recorded for each sample (camera gain 350; shutter speed 14.99 ms) and the sample was refreshed between each recording. Videos were analysed using NTA software (version 2.3) using automated settings for blur, threshold, and minimum particle size and minimum track length. Instrument calibration was verified by analyzing silica microspheres (Polysciences, Warrington, Pa.) prior to each analysis.

The protocol for immunoblotting was as follows: Microvesicles were quantitated using NTA, resuspended in LDS buffer and loaded on a NuPAGE 10-12% Bis-Tris gel (Invitrogen). The following primary antibodies were used: rabbit anti-flotilin (Abcam, 1:1000), rabbit anti-TSG 101 (Abcam 1:250), mouse anti-14-3-3 theta (Abcam, 1:250), rabbit anti-syntenin 1 (Abcam, 1:1000). Blots were visualized using HRP-conjugated secondary antibodies and the ECL Detection Reagent (Amersham).

### Initial Characterisation of the Exosome Proteome

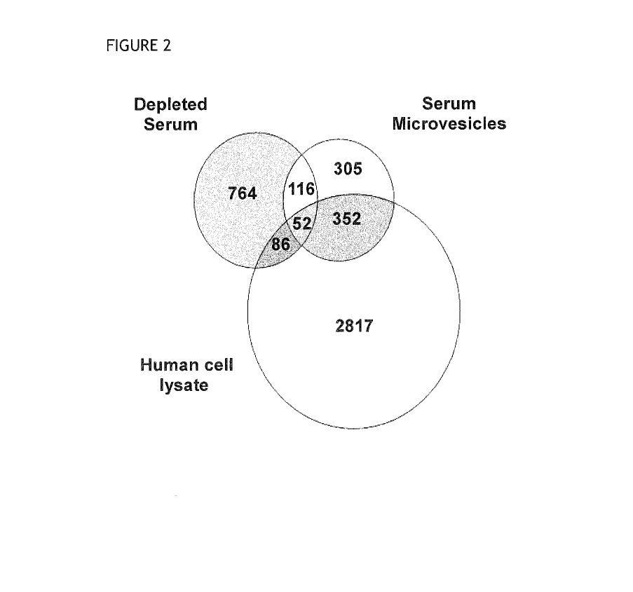

The proteins within the isolated microvesicles from the pooled NC (healthy) samples were quantified using label-free mass spectrometry (MS) to develop a “proteome profile” for the microvesicles. The specific enrichment of serum derived microvesicles enabled the identification and quantitation of proteins that are not detected in routinely processed serum samples. The profile obtained was then compared to the proteome of a pooled sample of albumin/immunoglobulin depleted serum (pool of 12 samples), and to the proteome of human cell lysates from HEK293 cells lysed in 1% NP40. The results are summarized in the Venn diagram in FIG. 2. Consistent with the notion that exosomes contain intracellular proteins, the microvesicles share a significant portion of their proteome with that identified in the cell lysates.

### Identification of Marker Proteins

The same mass spectrometric analysis was performed on patient samples using three biological replicates for idiopathic PD (3 groups each consisting of a pool of 12 different samples, total n=36), age- and co-morbidity matched controls (3 groups, total n=36) and one group of patients with motor neuron disease (MND), which is an unrelated neurodegenerative disease (n=22). Because PD is heterogeneous, it was also investigated whether changes that are detected in sporadic disease are also seen in patients with heterozygous mutations in GBA (n=13), which causes PD that is clinically and pathologically indistinguishable from sporadic cases.

Protein identification was based on at least three unique peptides identified with two technical replications. This LC-MS/MS analysis identified 619 proteins common to all groups, including bona fide exosome markers, with a false discovery rate of less than 1%.

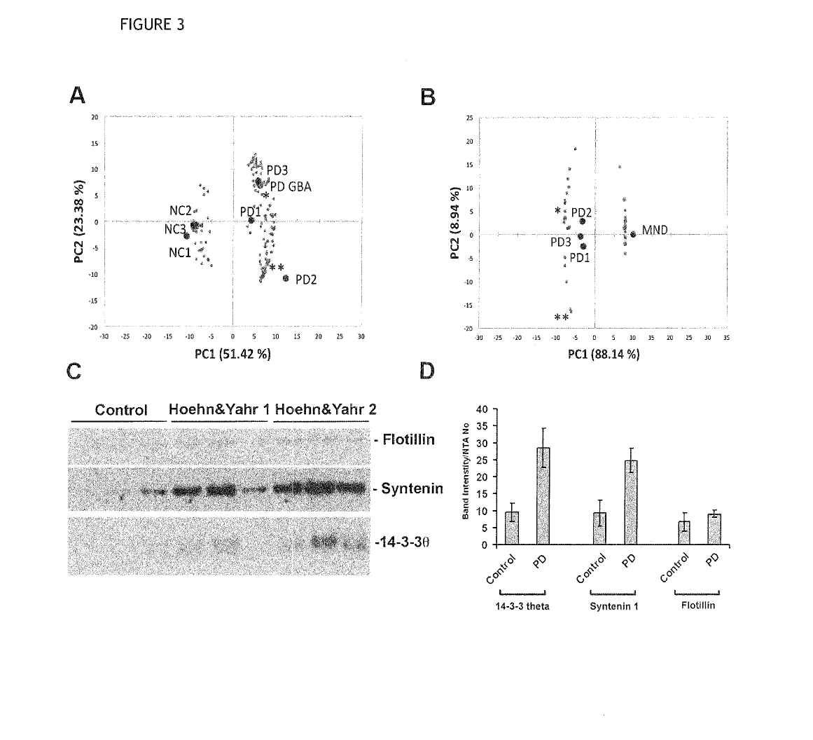

Label-free relative protein quantitation (Progenesis LCMS v4.1, nonlinear Dynamics) of the MS-identified proteins showed significant differential abundance in 143 proteins. Principal Component Analysis (PCA) (FIG. 3A) showed the separation of the analyzed samples (darker dots) and the loadings of the 143 proteins (lighter, smaller dots, * indicates syntenin 1, ** indicates 14-3-3θ). 38 proteins out of the 143 proteins shown in FIG. 3A can be used to separate PD and MND samples (ANOVA p<0.05, except 14-3-3 theta p=0.08—see FIG. 3B). The 38 proteins are listed in Table 3 below. Of the 38 proteins, 18 were identified as being associated with exosomes (shown in bold in Table 3 and listed in Table 1). Those 18 proteins are Syntenin-1, 14-3-3 theta, phosphoglycerate kinase 1 (PGK1), Programmed cell death protein 6, Complement factor B, Guanine nucleotide-binding protein G(i) subunit alpha-2, Fermitin family homolog 3, Alpha-2-macroglobulin, Integrin beta-1, Complement factor H, beta-actin, Glyceraldehyde-3-phosphate dehydrogenase, Casein kinase II subunit alpha, Prostaglandin E synthase 3, Glucose transporter type 1 (GLUT-1), POTE ankyrin domain family member J; 14-3-3 gamma, and Guanine nucleotide-binding protein G(k) subunit alpha. In the absence of significant changes in microvesicle number or size, these data demonstrate that either a subpopulation of serum microvesicles are differentially regulated or that certain proteins are enriched in microvesicles during the neurodegenerative process of PD. In either case, the 18 proteins identified have the potential to be markers for PD.

The protocol for the mass spectrometry experiments was as follows: Samples were prepared and analyzed on a LCMS system (Thermo LTQ Orbitrap Velos, Waters nAcquity) according to (Ref: Discovery of Candidate Serum Proteomic and Metabolomic Biomarkers in Ankylosing Spondylitis with minor changes). Briefly, proteins were proteolytically cleaved (Trypsin) after precipitation with Chloroform/Methanol (Ref: A method for the quantitative recovery of protein in dilute solution in the presence of detergents and lipids). Peptides were purified with C18 SepPac cartridges (Waters) and re-suspended in 0.1% TFA, 2% Acetonitrile before injection into the LC-MS system (Waters, nAcquity, 75 μm×250 mm, 1.7 μm particle size, Thermo LTQ Orbitrap Velos (60,000 Resolution, Top 20, CID) workflow and a gradient of 1-40% acetonitrile in 60 min at a flow rate of 250 nl/min. The serum sample was analyzed on a Q-Exactive LC-MS system (70,000 Resolution, Top 15, gradient and flow as above). Proteins were identified with Mascot (www.matrixscience.com) using a false discovery rate (FDR) of 1% and quantified with Progenesis LCMS (4.1).

TABLE 3

| | | Peptides | | | | Highest | Lowest | |
|---|---|---|---|---|---|---|---|---|
| | Peptide | used for | Confidence | Anova | Max fold | mean | mean | |
| Accession | count | quantitation | score | (p) | change | condition | condition | Description |
| O75340 | 2 | 2 | 97.76 | 8.66E−06 | Infinity | PD | MND | Programmed cell death protein 6 |
| P00751 | 18 | 18 | 1046.9 | 1.44E−05 | 1.981381 | MND | PD | Complement factor B |
| P10412 | 11 | 8 | 685.64 | 3.73E−05 | 2.797759 | PD | MND | Histone 1.4 |
| P07357 | 7 | 7 | 399.17 | 7.14E−05 | 5.451063 | MND | PD | Complement component C8 alpha chain |
| P04899 | 5 | 4 | 246.88 | 0.000167 | 3.171871 | PD | MND | Guanine nucleotide-binding protein G(i) |
| | | | | | | | | subunit alpha-2 |
| P07225 | 26 | 25 | 1900.33 | 0.000593 | 1.55354 | MND | PD | Vitamin K-dependent protein S |
| Q86UX7 | 6 | 6 | 300.57 | 0.000665 | 4.68863 | PD | MND | Fermitin family homolog 3 |
| P00558 | 2 | 2 | 92.28 | 0.000673 | 6.810956 | PD | MND | Phosphoglycerate kinase 1 (PGK1) |
| P01023 | 122 | 112 | 11284.88 | 0.000768 | 2.413314 | MND | PD | Alpha-2-macroglobulin |
| P08697 | 12 | 12 | 434.46 | 0.000823 | 3.77183 | MND | PD | Alpha-2-antiplasmin |
| P05556 | 10 | 10 | 411.1 | 0.00116 | 2.144397 | PD | MND | Integrin beta-1 |
| P10643 | 8 | 8 | 457 | 0.001301 | 2.166069 | MND | PD | Complement component C7 |
| P01876 | 22 | 9 | 1964.33 | 0.001451 | 2.113552 | MND | PD | Ig alpha-1 chain C region |
| O00560 | 13 | 13 | 1005.7 | 0.002641 | 1.922618 | PD | MND | Syntenin-1 |
| P08603 | 34 | 31 | 2234.8 | 0.002704 | 1.721309 | MND | PD | Complement factor H |
| P60709 | 24 | 8 | 1360.68 | 0.002713 | 3.464255 | PD | MND | Actin, cytoplasmic 1 |
| P04406 | 11 | 10 | 645.76 | 0.002751 | 1.883718 | PD | MND | Glyceraldehyde-3-phosphate dehydrogenase |
| | | | | | | | | (GAPDH) |
| P04003 | 44 | 42 | 3699.02 | 0.003182 | 1.778826 | MND | PD | C4b-binding protein alpha chain |
| P62979 | 8 | 8 | 507.1 | 0.005688 | 1.433756 | PD | MND | Ubiquitin-40S ribosomal protein S27a |
| P17844 | 3 | 3 | 118.01 | 0.005731 | 5.802019 | MND | PD | Probable ATP-dependent RNA helicase DDX5 |
| P00734 | 20 | 19 | 1359.98 | 0.005754 | 1.75746 | MND | PD | Prothrombin |
| P68400 | 3 | 2 | 111.17 | 0.005828 | 2.268366 | MND | PD | Casein kinase II subunit alpha |
| P0C0L4 | 103 | 2 | 8081.56 | 0.006904 | 1.737913 | MND | PD | Complement C4-A |
| P27105 | 12 | 12 | 816.18 | 0.008244 | 2.556389 | PD | MND | Erythrocyte band 7 integral membrane protein |
| P01814 | 2 | 1 | 89.36 | 0.011137 | 4.242204 | MND | PD | Ig heavy chain V-II region OU |
| P20851 | 6 | 6 | 403.75 | 0.01219 | 1.762433 | MND | PD | C4b-binding protein beta chain |
| P27635 | 2 | 2 | 98.39 | 0.013667 | 2.30189 | MND | PD | 60S ribosomal protein L10 |
| Q15185 | 2 | 2 | 70.81 | 0.015016 | 1.797969 | PD | MND | Prostaglandin E synthase 3 |
| P35125 | 2 | 1 | 59.8 | 0.015226 | 1.468813 | PD | MND | Ubiquitin carboxyl-terminal hydrolase 6 |
| P39060 | 3 | 3 | 152.25 | 0.016009 | 1.894222 | PD | MND | Collagen alpha-1(XVIII) chain |
| P11166 | 6 | 6 | 266.36 | 0.01612 | 2.935343 | PD | MND | Solute carrier family 2, (GLUT-1) |
| P78371 | 7 | 7 | 314.22 | 0.016768 | 1.786981 | PD | MND | T-complex protein 1 subunit beta |
| P01602 | 3 | 2 | 230.73 | 0.019197 | 3.278753 | MND | PD | Ig kappa chain V-I region HK102 |
| P0CG39 | 3 | 1 | 152.75 | 0.021103 | 1.934926 | MND | PD | POTE ankyrin domain family member J |
| P61981 | 4 | 2 | 171.88 | 0.029152 | 5.204093 | PD | MND | 14-3-3 gamma |
| P08754 | 2 | 1 | 94.71 | 0.081749 | 10.30251 | PD | MND | Guanine nucleotide-binding protein G(k) |
| | | | | | | | | subunit alpha |
| P27348 | 5 | 3 | 209.2 | 0.082457 | 1.939124 | PD | MND | 14-3-3 theta |
| P53618 | 2 | 2 | 44.65 | 0.11255 | 1.176336 | MND | PD | Coatomer subunit beta |

### Example 2

### Sample Populations

Serum samples were collected from the following groups of age-matched individuals:
NC: Controls: 3 groups of 10 per group,

mean age 67; 9 Male, 21 Female;

PD H&Y1: Idiopathic PD subjects at Hoehn-Yahr grade1: 3 groups of 10 per group,

mean age 66; 16 Male, 14 Female

PD H&Y2: Idiopathic PD subjects at Hoehn-Yahr grade2; 3 groups of 10 per group,

mean age 67; 18 Male, 12 Female

The samples were stored at −80° C. in accordance with standard protocols.

### Analysis of Syntenin 1 and 14-3-3 Theta Levels

Serum samples were pooled and microvesicles isolated as in Example 1. Immunoblotting experiments using antibodies specific for syntenin 1 and 14-3-3 theta were carried out on each pooled sample using the same protocol as in Example 1. The blots for each of the nine pooled samples are shown in FIG. 3C. The collated results for the 3× pooled H&Y2 samples as compared to the 3× pooled control samples are shown in FIG. 3D. The results confirm the initially identified changes in syntenin1 and 14-3-3 theta. H&Y2 patients have significantly higher levels of both proteins relative to controls (p<0.02 t-test).

### Example 3

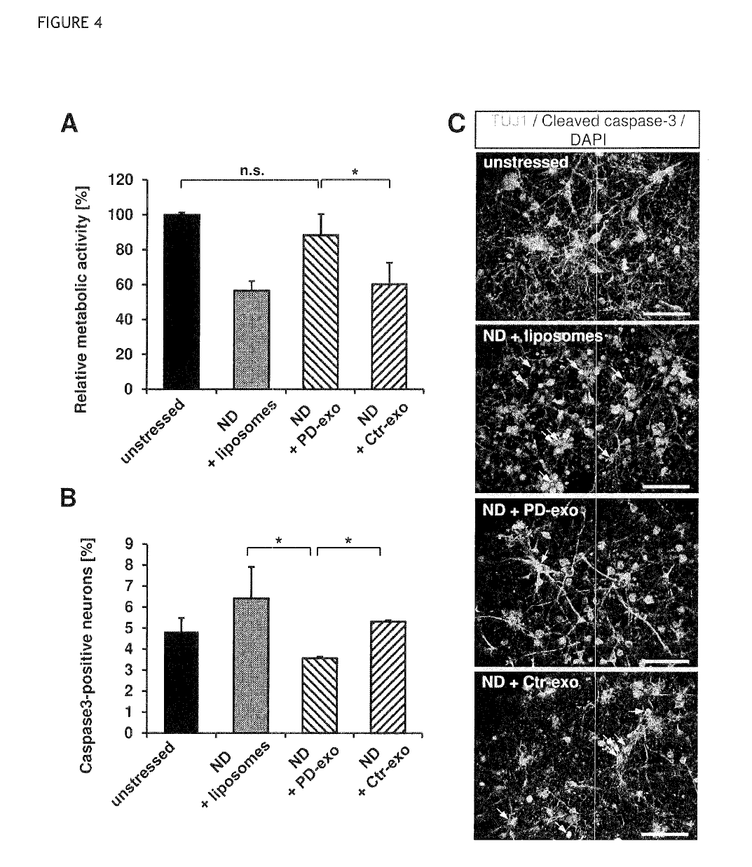

The enrichment of specific proteins in PD-derived microvesicles suggested that they may exhibit distinct biological properties. To investigate this hypothesis, a cell-based model was used to ask whether microvesicles derived from individual PD patients have a differential effect compared to those derived from healthy controls. Cultured day eight primary rat neurons were washed and left in nutrient deprived medium for a total of 24 h. Exosomes derived from individual patients (n=30 patients and 30 controls) or liposomes were added 5 h after nutrient deprivation. Since nutrient deprivation induces both exosome-endocytosis and oxidative stress (Kirchhoff et al., 2013), this model was used to investigate the effect of purified microvesicles or liposomes on neuronal viability. Strikingly, it was found using the MTT assay that the metabolic activity of neurons treated with PD-derived exosomes was significantly improved when compared to ones derived from age-matched controls or synthetic liposomes (FIG. 4A). To corroborate these findings using an alternative readout of toxicity, microvesicle or liposome-treated cells were stained and quantified in an unbiased fashion for caspase 3 activation, which is an indicator of apoptosis. It was found that wells treated with PD-derived exosomes had significantly less activated caspase 3 positive neurons compared to liposome treated or controls (FIG. 4 panels B,C). Collectively, these data indicate that circulating microvesicles in PD have a distinct biochemical composition and convey a neuroprotective effect.

The protocol for the preparation of cortical neurons was as follows: All experiments were conducted in accordance with institutional and governmental guidelines. Primary cortical neurons were prepared from PO rats. After brain dissection and removal of the meninges and blood vessels, the cortical tissue was trypsinized. The cortical tissue was washed with Minimal Essential Medium (MEM, supplemted with FCS and PSA; Gibco) and was then triturated to yield a single cell suspension followed by centrifugation for 5 minutes at 1100 g. The medium was discarded and 5 ml of fresh MEM medium was applied to the cells followed by a second trituration. Cells were seeded at appropriate density onto PLL-coated cell culture plates. MEM was completely replaced with neurobasal/B27 (both, Gibco) 2-3 h after seeding. Cortical neurons (CN) were incubated at 37° C., 5% CO2. Half of the medium was replaced every 3 days and mitotic inhibitor (2 μM cytosine β-D-arabinofuranoside [araC], Sigma) was added during the first media exchange to arrest glial growth.

The protocol for the immunofluorescence staining of cortical neurons was as follows: Cortical neurons (CN, 1.5×10⁵ cells/24-well) were cultured for 7 days in neurobasal/B27 medium at 37° C. For immunofluorescent staining, cortical neurons plated on cover slips were fixed with 4% PFA, washed with 0.1% PBS and permeabilised with PBS containing 0.5% Tween-20 (0.5% PBST) and blocked with 1% (w/v) BSA (Sigma). The following primary antibodies were incubated overnight at 4° C.: mouse anti-β-III tubulin (Convance, 1:1000), rabbit anti-cleaved caspase-3 (CellSignal, 1:400), rabbit anti-syntenin 1 (Abeam, 1:2000), and mouse anti-14-3-3theta (Abeam, 1:1000). Neurons were washed with 0.1% PBST and incubated under light-protected conditions with the appropriate secondary antibodies: goat anti-mouse AlexaFluor488 and goat anti-rabbit AlexaFluor568 (both Invitrogen, 1:500). Neurons were mounted in DAKO mounting medium containing DAPI. Cells were analysed with a Leica DM2500 fluorescence microscope (Leica). For quantification of caspase-3 positive neurons, digital photographs of five random fields were taken with a camera (Rotera XR Fast 1394, QImaging) and the percentage of caspace-3 positive, β-III tubulin positive neurons were counted in an unbiased manner. All experiments were repeated at least three times.

The protocol for the neuronal viability assays was as follows: Primary cortical neurons (CN, 4×10⁴/96-well) were cultured for seven days in neurobasal medium supplemented with B27. For nutrient deprivation (ND), neurons were cultured for 5 h in medium lacking B27 supplement before exposed to exosomes or liposomes. Isolated exosomes or liposomes were applied in ND medium and incubated for further 16 h. Neuronal viability was assayed by MTT assay: 0.5 mg/ml 3-(4,5-dimethylthiazol-2-yl)-2,5-diphenyltetrazoliumbromide (MTT, Sigma) was dissolved in ND medium and added to the CN for 2 h. Formazan crystals were solubilised in DMSO and absorbance was measured at 570 nm using a plate reader (FLUOstar Optima, BMG Labtech). Liposomes (Liposome preparation kit, Sigma) were prepared by rotatory evaporation and resuspended in HBSS. BSA or recombinant 14-3-3theta protein were diluted at 20 nM in HBSS and then used to resuspend the lipid bilayer to yield liposomes. The number of exosomes and liposomes was determined by NTA analysis and equal numbers of vesicles were added to CN (800 exosomes/neuron, 24-well; 80 exosomes/neuron, 96-well).

1. A method for the identification or monitoring of Parkinson's Disease (PD) in an individual, which method comprises measuring the level of at least one marker selected from the proteins shown in Table 1 in a first sample taken from the individual, wherein if the level of 14-3-3 theta is measured said sample is not taken from the cerebral cortex of the individual.

2. A method according to claim 1, which method comprises comparing the level of said at least one marker in said first sample to a control level and:
identifying the individual as having PD if the level of said at least one marker in said first sample is higher than said control level; or
identifying the individual as not having PD if the level of said at least one marker in said first sample is lower than said control level;
and optionally administering a therapeutically effective amount of a treatment agent to an individual identified as having PD by the said method.

3. A method according to claim 1, which method comprises comparing the level of said at least one marker in said first sample to the level of said at least one marker in a second sample taken from the same individual at a later time and:
identifying the PD of the individual as worsening if there is a higher level of said at least one marker in said second sample relative to said first sample, or
identifying the PD of the individual as improving if there is a lower level of said at least one marker in said second sample relative to said first sample.

4. A method according to claim 2, wherein the control level of said at least one marker is the level of said marker measured in a sample taken from an individual not suffering from Parkinson's disease, wherein if the level of 14-3-3 theta is measured said sample is not taken from the cerebral cortex of the individual.

5. A method according to claim 1, in which a level of said at least one marker in a said sample is determined to be higher than a level to which it is compared if it is at least 1.1-fold, 1.2-fold, 1.5-fold, 1.75 fold, 2-fold, 3-fold, 4-fold, 5-fold or 10-fold higher than the level to which it is compared, and a level of said at least one marker in a said sample is determined to be lower than a level to which it is compared if it is at least 1.1-fold, 1.2-fold, 1.5-fold, 1.75 fold, 2-fold, 3-fold, 4-fold, 5-fold or 10-fold lower than the level to which it is compared.

6. A method according to claim 1, wherein a said sample comprises a biological fluid selected from blood, serum, urine and cerebrospinal fluid.

7. A method according to claim 6, wherein a said sample is a serum sample.

8. A method according to claim 1, which method comprises measuring the said at least one marker in exosomes isolated from a said sample.

9. A method according to claim 8 wherein said exosomes are isolated from said sample by centrifugation or a microfluidic device

10. A method according to claim 2, wherein the individual is suspected of having a neurodegenerative disorder which may or may not be PD.

11. A method according to claim 10, wherein the neurodegenerative disorder which is not PD is motorneurone disease (MND), Alzheimers Disease (AD), Multisystem atrophy or Fronto-temporal Dementia (FTD).

12. (canceled)

13. A method for determining the effect of a treatment agent on the progression of PD, which method comprises monitoring the progression of PD in an individual in accordance with a method as defined in claim 3, wherein the first sample is taken from the individual prior to the administration of a treatment agent and the second sample is taken from the individual after the administration of a treatment agent.

14. A method according to claim 13, further comprising:
if the PD of the individual is identified as worsening, increasing the dose of the treatment agent and/or administering an alternative treatment agent; or
if the PD of the individual is identified as improving, decreasing the dose of the treatment agent and/or administering an alternative treatment agent with a lower risk of adverse side-effects.

15. A method according to claim 2 or 13 wherein the treatment agent is selected from L-Dopa, an anti-apoptotic, an anti-oxidant, an anti-glutamatergic, a monoamine oxidase B inhibitor, an adenosine antagonist, a dopamine agonist, a mitochondrial stabiliser, a promoter of alpha-synuclein clearance and a trophic factor.

16. A method according to claim 1, wherein the at least one marker is selected from Syntenin-1, 14-3-3 theta and phosphoglycerate kinase 1 (PGK1), and is preferably Syntenin-1.

17. A method for the treatment of PD in an individual, which method comprises administering to the individual a composition comprising microvesicles, wherein said microvesicles contain at least one protein of Table 1, or which method comprises stimulating the endogenous production of said microvesicles.

18-19. (canceled)
